(19) United States
(12) Patent Application Publication
(10) Pub. No.: US 2023/0085355 A1
(43) Pub. Date: Mar. 16, 2023

(54) MICROFLUIDIC COMPONENT USED FOR MEASURING ELECTRICAL IMPEDANCE ACROSS A BIOLOGICAL OBJECT

(71) Applicant: Commissariat à l'Energie Atomique et aux Energies Alternatives, Paris (FR)

(72) Inventors: Marie-Line COSNIER, Grenoble Cedex 9 (FR); Nicolas VERPLANCK, Grenoble Cedex 9 (FR); Manuel ALESSIO, Grenoble Cedex 9 (FR); Anubhav BUSSOOA, Grenoble Cedex 9 (FR); Pascal MAILLEY, Grenoble Cedex 9 (FR); Frédéric REVOL-CAVALIER, Grenoble Cedex 9 (FR); Jean-Maxime ROUX, Grenoble Cedex 9 (FR)

(73) Assignee: Commissariat à l'Energie Atomique et aux Energies Alternatives, Paris (FR)

(21) Appl. No.: 17/931,308

(22) Filed: Sep. 12, 2022

(30) Foreign Application Priority Data
Sep. 13, 2021 (FR) 21 09560

(51) Int. Cl. B01L 3/00; G01N 15/10

(52) U.S. Cl. CPC B01L 3/502715; G01N 15/1031; G01N 2015/0065

(57) ABSTRACT

A microfluidic component used for measuring electrical impedance across a biological object, the component including a microfluidic space including a zone referred to as measurement zone, at least two electrodes arranged facing one another on each side of the measurement zone, the component being formed by assembling, along a longitudinal junction plane, at least two superposed layers referred to as lower layer and upper layer, the two layers each having at least one cavity, the two layers being assembled with one another in such a way as to position the two cavities facing one another in order to form the microfluidic space.

### TECHNICAL FIELD OF THE INVENTION

The present invention relates to a microfluidic component used for measuring electrical impedance across a biological object, it being possible for this component to be used to characterize a biological object such as a spheroid or to count biological objects present in a fluid flow.

### PRIOR ART

Studies are currently being conducted in an attempt to better characterize a biological object such as a spheroid formed of a cluster of biological cells. The characterization relates notably to the viability of the biological cells that make up the spheroid. Studies have shown that there may be a correlation between the viability of the cells present in the spheroid and the electrical impedance measured across the spheroid. In other words, the greater the electrical impedance measured across the spheroid, the more living cells the spheroid might contain. Even though this relationship has not yet been fully established, numerous studies are now attempting to demonstrate it. These studies rely on the use of microfluidic devices for measuring electrical impedance. Notable mention may be made of the following studies:
Wu_2018—Electrical impedance tomography for real-time and label-free cellular viability assays of 3D tumour spheroids—Analyst/University of Edinburgh.
Viswam_2018—Impedance Spectroscopy and Electrophysiological Imaging of cells with a high-density CMOS microelectrode array system—IEEE Transactions on biomedical circuits and systems/Ecole polytechnique de Zurich (ETH)
Heileman_2015—Microfluidic platform for assessing pancreatic islet functionality through dielectric spectroscopy—Biornicrofluidics/McGill University, Montreal

The various devices used in these studies are not, however, satisfactory, for the following reasons:
The electrodes used are coplanar and the biological objects that are to be characterized are either in contact with the electrodes, or too far away. In certain cases, the field lines produced between the electrodes do not pass optimally through the biological objects that are to be characterized.
The materials used for incorporating the electrodes into these fluidic systems are often not transparent (the materials of the electrodes or of the chamber) or the configuration of the devices is not suitable for viewing and monitoring the biological object. It is often impossible to set up transmission microscopy monitoring even though this method of observation is standard practice in biology.

Patent U.S. Pat. No. 8,454,813 B2 describes a cell sorting device (cytometer). The purpose of that device is not to characterize a biological object through measurements. Indeed the electrodes are positioned in such a way that the field created presses the cell against the bottom of the well.

Patent application US2010/270176A1 itself describes a device for characterizing neurons. That device uses coplanar electrodes arranged at the bottom of the cavity. That solution makes it possible to press the biological object against the bottom of the cavity, something which is not optimal for characterizing it through measurements.

It is an object of the invention to propose a microfluidic component that allows a biological object to be characterized and that is:
easy to manufacture;
suitable for making reliable measurements of electrical impedance across the biological object;
suitable also for performing optical monitoring, for example using a transmission microscope.

The microfluidic component needs to have a structure in which the biological object can be raised clear of the bottom of the cavity so that the field lines generated between the two electrodes can thus pass through said object.

### SUMMARY OF THE INVENTION

This object is achieved by means of a microfluidic component used for measuring electrical impedance across a biological object, said component comprising:
a microfluidic space comprising a zone referred to as measurement zone,
at least two electrodes arranged facing one another on each side of the measurement zone,
the component being formed by assembling, along a longitudinal junction plane, at least two superposed layers referred to as lower layer and upper layer,
the two layers each having at least one cavity,
the two layers being assembled with one another in such a way as to position the two cavities facing one another in order to form said microfluidic space,
the two cavities having cross sections of different sizes, forming two clearance surfaces, one on each side of said microfluidic space,
two electrically conducting deposits being applied to said two clearance surfaces so as to form said two electrodes.

According to one particular embodiment, the microfluidic space comprises a hydrodynamic trap forming said measurement zone.

According to another particular embodiment, the upper layer and/or the lower layer has a transparent part situated facing the measurement zone.

According to another particular embodiment, the microfluidic space is produced in the form of a canal hollowed into said microfluidic component.

According to another particular embodiment, the hydrodynamic trap is produced in the form of a step arranged inside said canal, downstream of the measurement zone.

According to another particular embodiment, the hydrodynamic trap is produced in the form of one or more posts arranged inside said canal, downstream of the measurement zone.

According to another particular embodiment, the microfluidic space is produced in the form of a well hollowed into said microfluidic component.

According to another particular embodiment, the lower layer and/or the upper layer is made from a material selected from cyclic olefin copolymer, polymethyl methacrylate, and an assembly of silicon and of glass.

According to another particular embodiment, each electrically conducting deposit is made from a metallic material or in the form of a conducting ink.

The invention relates to a system for measuring electrical impedance across a biological object, comprising a potentiostat comprising two connection terminals, said system comprising a microfluidic component as defined hereinabove, of which the two electrodes are each connected to a distinct terminal of the potentiostat.

The invention relates to a method for manufacturing a microfluidic component as defined hereinabove, the method comprising the steps of:
creating a first cavity in the lower layer,
depositing a conducting layer in at least two distinct zones of the lower layer, on each side of the first cavity,
creating a second cavity in the upper layer, said first cavity and said second cavity being created with distinct cross sections so as to create two clearance surfaces which are occupied by the conducting layer,
assembling the lower layer and the upper layer by placing the first cavity and the second cavity to face one another so as to create said microfluidic space, the conducting layer being arranged between said lower layer and said upper layer.

According to one particular feature, the method comprises a step of creating a hydrodynamic trap in the microfluidic space.

It may be noted that the component of the invention may notably be produced from a minimum of layers. The two electrodes are raised clear of the bottom of the cavity so that the field lines can pass right through the biological object.

### BRIEF DESCRIPTION OF THE FIGURES

Further features and advantages will become apparent from the following detailed description given with reference to the attached drawings in which:

FIG. 1 illustrates, viewed in cross section, the architecture of the microfluidic component of the invention;

FIG. 2 depicts a view from above of the microfluidic component of the invention, according to a first embodiment variant;

FIG. 3 depicts a view from above of the microfluidic component of the invention, according to a second embodiment variant;

FIG. 4 illustrates the principle of transmission optical observation of the biological object using this architecture;

FIG. 5 illustrates the principle of manufacture of the microfluidic component of the invention;

FIG. 6 illustrates a first principle for creating the hydrodynamic trap used in the microfluidic component of the invention;

FIG. 7 illustrates a second principle in the creation of the hydrodynamic trap used in the microfluidic component of the invention;

FIG. 8 is a diagram showing the variation in impedance measured across the growth medium, across a spheroid trapped in the measurement zone, and across two spheroids trapped in the measurement zone.

### DETAILED DESCRIPTION OF AT LEAST ONE EMBODIMENT

In the following part of the description, the terms “lower”, “upper”, “above”, “below” or equivalent are to be considered with regard to the position of the microfluidic component on a horizontal support.

The term “longitudinal” is to be understood in directions parallel to the horizontal support and the term “transverse” in directions perpendicular to the horizontal support.

The invention seeks to make it possible to measure the electrical impedance across a biological object.

The biological object is, for example, a cluster of cells. What is meant according to the invention by a cluster of cells is the three-dimensional auto assembly of one or more cell types. Such a cluster of cells may notably be referred to as a spheroid, an organoid, a neurosphere. This cluster may also be an islet of Langerhans. In the remainder of the description, the term “spheroid”, referenced S, will be used generically to evoke such a cluster, that term being the one conventionally employed in the field of living-cell culture. Nonlimitingly, such a spheroid S may for example have a diameter ranging from a few tens of pm to a few hundred pm.

The microfluidic component 1 of the invention may take the form of a support in which there is formed a microfluidic space 10, of non zero volume, suitable for the presence of a spheroid S. This microfluidic space 10 may take the form of a canal, of a well or equivalent.

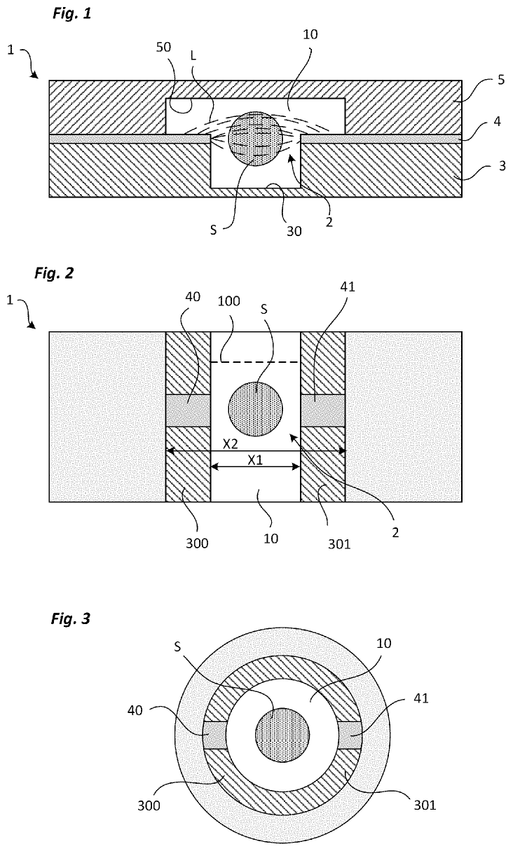

FIG. 1 and FIG. 2 show a microfluidic space 10 produced in the form of a canal. FIG. 3 shows a microfluidic space 10 produced in the form of a well. The microfluidic space may be closed at the top and at the bottom by the assembling of the layers (see below).

The microfluidic space 10 comprises a hydrodynamic trap 100 designed to trap the spheroid S and hold it in a stable position defining a measurement zone 2 in which the impedance measurements can be taken. It will be seen hereinbelow that this hydrodynamic trap 100 may be produced according to different embodiment variants, via a mechanical and/or fluidic solution.

As depicted in FIG. 1, the microfluidic component 1 is formed by assembling several superposed layers 3, 4, 5. The assembling of the layers makes it possible to create said microfluidic space 10 as well as the electrodes 40, 41 needed for measuring the electrical characteristics of the spheroid S. This microfluidic space 10 is intended to receive a liquid in which the spheroid S that is to be electrically characterized is placed.

The liquid is advantageously chosen so that the spheroid remains lifted clear of the bottom of the trap and does not touch said bottom. It may notably have a composition of sufficient viscosity to keep the spheroid at a height and so that it does not touch the bottom of the trap.

The component thus comprises:
a lower layer 3 in which a first cavity 30 is produced;
a conducting layer 4 deposited in at least two distinct zones of the upper face of the lower layer 3, the two zones being separated from one another;
an upper layer 5 in which a second cavity 50, greater in size than the first cavity 30, is produced.

Upon assembly, the first cavity 30 and the second cavity 50 are brought to face one another so as to form said microfluidic space 10. What is meant by a cavity is a non-emergent hole produced in the layer concerned. Each layer (lower and upper) may be produced by the superposition of several layers/strata. By bringing the two cavities to face one another, the microfluidic space 10 is closed at the top and at the bottom.

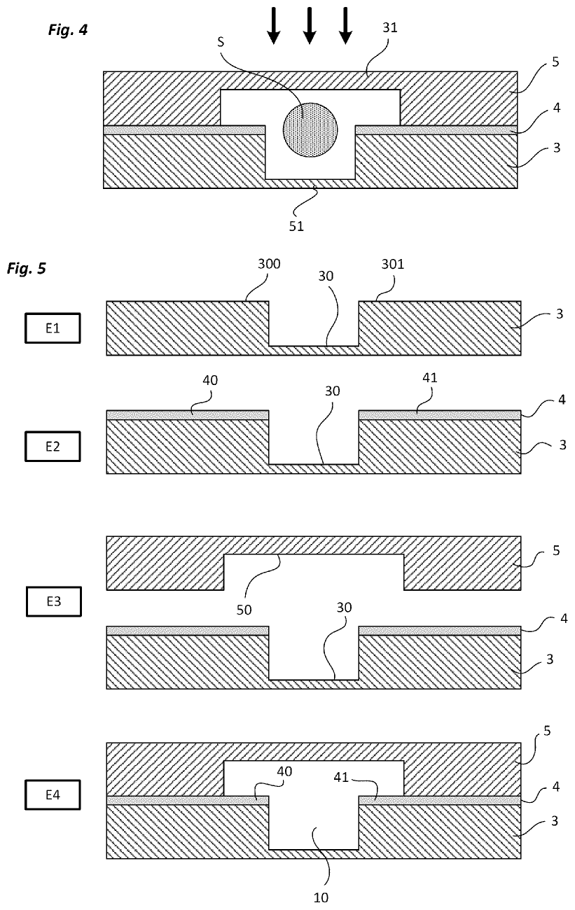

Advantageously, as depicted in FIG. 4, the lower layer 3 and/or the upper layer 5 each comprise at least one transparent zone 31, 51, these zones being situated one above the other, along the axis of the microfluidic space, to create a viewing window through the lower layer and the upper layer of the support so that monitoring can be performed, through the microfluidic space 10, for example using transmission microscopy.

The lower layer 3 and the upper layer 5 may be made from a transparent material such as COC (cyclic olefin copolymer), PMMA (polymethyl methacrylate) or equivalent. The upper layer 5 may also be manufactured by assembling two strata, a lower stratum made of silicon and an upper stratum made of glass. Of course other variants could be envisioned.

According to one particular aspect of the invention, the two cavities 30, 50 have cross sections distinct from one another, over at least part of their length, so as to create two advantageously coplanar clearance surfaces 300, 301 situated in the junction plane along which the two layers are joined on each side of the measurement zone 2 of the microfluidic space 10. In the figures, the cross section of the cavity 30 of the lower layer 3 is chosen smaller than that of the cavity 50 of the upper layer 5. It should be noted that the cross sections of the two cavities 30, 50 are not necessarily constant over the entire length of the microfluidic component 1. They are just distinct at the measurement zone in order to create the two clearance surfaces 300, 301.

Each clearance surface 300, 301 comprises a distinct deposition zone of the conducting layer 4 so as to create a distinct electrode 40, 41 in each zone. By creating the two clearance surfaces 300, 301, the electrodes find themselves in contact with the liquid present in the microfluidic space, making it possible to create field lines L between them, passing through the spheroid S present in the measurement zone 2.

By way of example and nonlimitingly, with a microfluidic space 10 produced in the form of a canal, the cavity 30 produced in the lower layer 3, that forms a first part of this canal, may have a width X1 of between 200 and 500 μm and the cavity 50 produced in the upper layer 5 to form the second part of the canal may have a width X2 of between 300 and 700 μm, in the knowledge that the objective is to contrive for X2 to be greater than X1 in order to create the two clearance surfaces that are intended to at least partially accommodate the two electrodes, one on each side of the measurement zone 2. Ideally, the two electrodes have the same surface area. Their length is therefore (X2-X1)/2. The electrodes may for example have a width of between 100 μm and 400 μm.

The electrodes 40, 41 are preferably metallic and produced by vaporization after masking, or using a “lift-off” technique. The materials deposited may be dependent on the application (resistivity, biocompatibility, etc.) but the materials conventionally used are gold (with a titanium or chromium sublayer) or platinum. A variant may be to use conducting inks deposited for example using screen printing, inkjet, spray.

The two electrodes 40, 41 are thus separated by the cross section of the cavity 30 that defines the measurement zone 2, upstream of the hydrodynamic trap 100 intended to block the spheroid S. The spheroid S is brought into the measurement zone 2 to be subjected to the measurements of impedance between the two electrodes.

The two electrodes 40, 41 may partially occupy one of the two clearance surfaces created around the microfluidic space 10 as a result of the difference in cross section between the cavity in the upper layer 5 and the one in the lower layer 3. They may extend into the junction between the two layers, lower layer 3 and upper layer 5, to allow electrical contact to be picked up at a distance.

According to one particularly advantageous aspect, as the electrodes 40, 41 are arranged in a longitudinal plane, it is possible to observe the spheroid S present in the microfluidic space 10 by transparency through the upper layer 5 and the lower layer 3 of the support of the component 1.

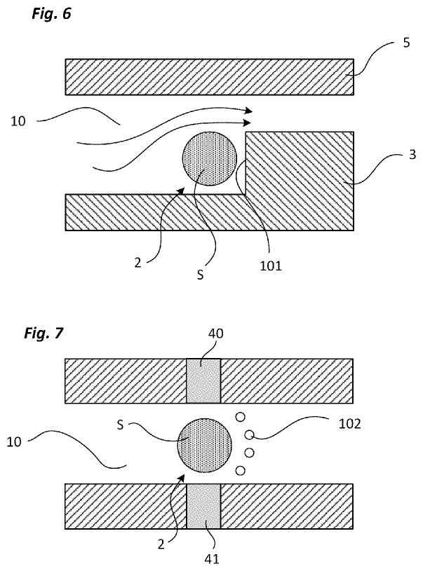

With reference to FIGS. 6 and 7, a hydrodynamic trap 100 is thus created in order to position the spheroid S between the two electrodes 40, 41 in the microfluidic space 10. A first variant depicted in FIG. 6 consists in creating a step 101 in the microfluidic space 10. By way of example, FIG. 6 shows a microfluidic space 10 produced in the form of a canal. A step 101 is inserted inside the canal, forming an end stop for the spheroid S as it circulates along said canal.

FIG. 7 shows a second embodiment of the hydrodynamic trap 100, formed by posts 102 arranged in the microfluidic space 10 to block the spheroid S and keep it in the measurement zone.

In another embodiment variant which has not been depicted, the canal comprises a restriction, situated downstream of the measurement zone arranged between the two electrodes, this restriction forming the hydrodynamic trap 100. The restriction is tight enough to block the spheroid S and keep it in the measurement zone.

In another variant which has not been depicted, the hydrodynamic trap 100 may also be produced using a gel present in the microfluidic space 10, at the measurement zone. The spheroid S is injected into the microfluidic space 10 and held in position in the measurement zone by gelification. This variant is also compatible with the trapping solutions of a mechanical nature which have been described hereinabove.

It should be noted that the microfluidic component 1 of the invention is associated with a potentiostat, to the terminals of which each electrode 40, 41 of the component 1 is connected.

The assembling of the various layers 3, 4, 5 will depend on the materials but all the solutions for assembling the materials mentioned hereinabove are conceivable, particularly thermocompression, screen printing, laser welding, adhesives, ultrasound or even direct and anodic bonding, in the case of parts containing silicon and glass.

The microfluidic component 1 may notably be used to count biological objects in a flow. To do that, it is necessary to detect the variations in electrical impedance in the measurement zone, each significant variation corresponding to the passage of one distinct biological object.

FIG. 5 illustrates the various steps of the method for manufacturing the microfluidic component 1 of the invention:

E1: The lower layer 3 is prepared, in order to create the cavity 30 therein. This cavity may be produced by micromachining, thermoforming, hot pressing, injection molding or even chemical or dry etching in the case of silicon or glass, or any other technique that enables the creation of this type of cavity. The two clearance surfaces 300, 301 are present on each side of the cavity 30. Ideally, the roughness of these surfaces is very low (polished, or even optical quality) in order to optimize the adhesion of the layer 4 and the assembly with the upper layer 5.

E2: A conducting layer 4 is deposited on the upper face of the lower layer 3, in two distinct zones one on each side of the cavity 30. The two electrodes formed at least partially occupy the two clearance surfaces 300, 301.

E3: The upper layer 5 is prepared to create its cavity 50. This cavity may be produced using the methods listed hereinabove in respect of the cavity 30. For the one same microfluidic component 1, two distinct methods may likewise be used for producing the cavities 30, 50. The cavity 50 is brought against the lower part formed of the lower layer 3 and of the conducting layer 4. The two cavities are brought to face one another so as to create the microfluidic space 10. The two electrodes 40, 41 are at least partially present on the two clearance surfaces 300, 301 created as a result of the difference in cross section between the two cavities 30, 50.

E4: The microfluidic component 1 is assembled and ready for use.

The various above-described manufacturing steps can be adapted to suit other architectures of component. It should also be noted that the hydrodynamic trap 100 can be created in the first step E1, during the creation of the cavity 30, or in the third step E3, during the creation of the cavity 50.

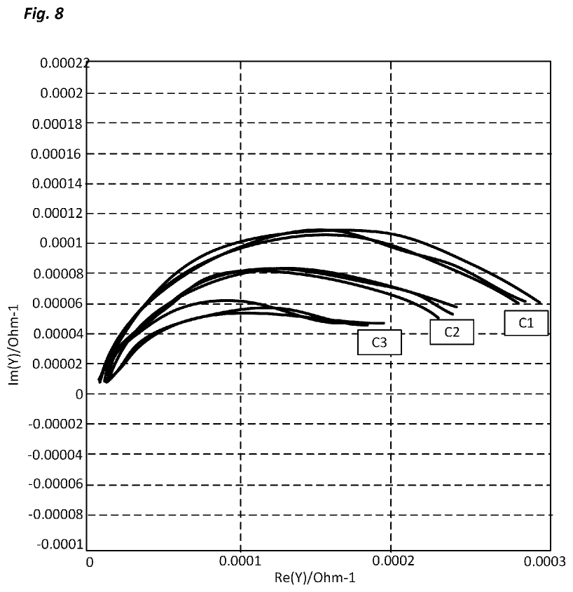

The diagram of FIG. 8 represents the impedance Im (in the form of a Nyquist diagram) measured when the fluidic canal is full of culture medium only (curves 01), when one spheroid S is trapped by the step arranged between the electrodes (curves C2) in the measurement zone 2, and when two spheroids S are trapped by the step arranged between the electrodes (curves C3) in the measurement zone 2.

The solution of the invention offers numerous advantages, including:
it allows the electrical impedance across a biological object to be measured in a simple and reliable way while at the same time maintaining monitoring through the transparent parts of the support;
it is simple to manufacture, by assembling layers, using conventional manufacturing techniques;
it is low cost.

1. A microfluidic component used for measuring electrical impedance across a biological object, said component comprising:
a microfluidic space comprising a zone referred to as measurement zone,
at least two electrodes arranged facing one another on each side of the measurement zone,
the component being formed by assembling, along a longitudinal junction plane, at least two superposed layers referred to as lower layer and upper layer,
the two layers each having at least one cavity,
the two layers being assembled with one another in such a way as to position the two cavities facing one another in order to form said microfluidic space,
wherein:
the two cavities have cross sections of different sizes, forming two clearance surfaces, one on each side of said microfluidic space,
two electrically conducting deposits are applied to said two clearance surfaces so as to form said two electrodes.

2. The microfluidic component as claimed in claim 1, wherein the microfluidic space comprises a hydrodynamic trap forming said measurement zone.

3. The microfluidic component as claimed in claim 1, wherein the upper layer and/or the lower layer has a transparent part situated facing the measurement zone.

4. The microfluidic component as claimed in claim 1, wherein the microfluidic space is produced in the form of a canal hollowed into said microfluidic component.

5. The microfluidic component as claimed in claim 4, wherein the hydrodynamic trap is produced in the form of a step arranged inside said canal, downstream of the measurement zone.

6. The microfluidic component as claimed in claim 4, wherein the hydrodynamic trap is produced in the form of one or more posts arranged inside said canal, downstream of the measurement zone.

7. The microfluidic component as claimed in claim 1, wherein the microfluidic space is produced in the form of a well hollowed into said microfluidic component.

8. The microfluidic component as claimed in claim 1, wherein the lower layer and/or the upper layer is made from a material selected from cyclic olefin copolymer, polymethyl methacrylate, and an assembly of silicon and of glass.

9. The microfluidic component as claimed in claim 1, wherein each electrically conducting deposit is made from a metallic material or in the form of a conducting ink.

10. A system for measuring electrical impedance across a biological object, comprising a potentiostat comprising two connection terminals, wherein said system comprises a microfluidic component as defined in claim 1, of which the two electrodes are each connected to a distinct terminal of the potentiostat.

11. A method for manufacturing a microfluidic component as defined in claim 1, wherein said method comprises steps of:
creating a first cavity in the lower layer,
depositing a conducting layer in at least two distinct zones of the lower layer, on each side of the first cavity,
creating a second cavity in the upper layer, said first cavity and said second cavity being created with distinct cross sections so as to create two clearance surfaces which are occupied by the conducting layer,
assembling the lower layer and the upper layer by placing the first cavity and the second cavity to face one another so as to create said microfluidic space, the conducting layer being arranged between said lower layer and said upper layer.

12. The method as claimed in claim 11, wherein said method comprises a step of creating a hydrodynamic trap in the microfluidic space.
